(19) United States
(12) Patent Application Publication
(10) Pub. No.: US 2013/0068242 A1
(43) Pub. Date: Mar. 21, 2013

(54) Semi-Permanent Mascara and Method of Applying

(71) Applicant: Cry Baby Culture, LLC, Phoenix, AZ (US)

(72) Inventor: Jennifer Green, Phoenix, AZ (US)

(73) Assignee: Cry Baby Culture, LLC, Phoenix, AZ (US)

(21) Appl. No.: 13/678,251

(22) Filed: Nov. 15, 2012

Related U.S. Application Data
(63) Continuation-in-part of application No. 12/539,611, filed on Aug. 12, 2009, now abandoned.
(60) Provisional application No. 61/188,656, filed on Aug. 12, 2008.

(51) Int. Cl. A61K 8/81; A61K 8/02; A61Q 1/10

(52) U.S. Cl. CPC A61K 8/8164; A61Q 1/10; A61K 8/02; A61K 8/8123; A61K 8/8135
USPC 132/209; 424/70.7; 424/401; 132/202

(57) ABSTRACT

A semi-permanent mascara which includes a pharmaceutical grade cyanoacrylate that is mixed with a small amount of irregularly-shaped, micro- or nano-sized polytetrafluoroethene fibers, and then professionally applied to the eyelashes creating a semipermanent mascara application that is 100% waterproof, will not run, smear, or smudge, and lasts 2 to 3 weeks.

### CROSS-REFERENCE TO RELATED APPLICATIONS

This application is a continuation-in-part and claims the benefit of U.S. patent application Ser. No. 12/539,611, filed Aug. 12, 2009, which is a non-provisional and claims the benefit of U.S. Pat. App. Ser. No. 61/188,656, filed Aug. 12, 2008.

### FIELD OF INVENTION

This invention relates generally to cosmetics. This invention relates more specifically to semi-permanent eye makeup and a process of applying it.

### BACKGROUND

There are many cosmetic compositions and methods of application for thickening, coloring, darkening, curling, and lengthening eyelashes. Mascara is a common composition known to thicken, darken, color, and curl the lashes, but with significant drawbacks. Traditional mascara runs and smears very soon after application, lasting only a day or less before it must be removed and reapplied. This problem is exacerbated when traditional mascara is exposed to water or excessive humidity. Exercising, swimming, and crying are all activities sure to turn the mascara into a runny mess. Throughout the years, different chemicals and additives have been created in an attempt to make the mascara more waterproof and smudgeproof. However, mascara claiming to be waterproof still only lasts 24 hours and can be smudged or removed when contacted with sunscreen creams and oils. A mascara that remains in place for an extended time without the need to reapply, and that withstands exposure to moisture is needed. It would further be advantageous to combine mascara with a lengthening agent into a single composition, as mascara may add volume and color but does not lengthen the lashes.

Other approaches also have significant drawbacks. Tinting, coloring or dyeing the eyelashes with tints or dyes only adds pigment, not volume, texture or length. Tinting, coloring or dyeing the eyelashes is also not permitted in all states. Strip lashes are temporarily glued onto the skin and fall off easily and can not endure any water. Eyelash extensions use an adhesive to adhere one synthetic lash to the existing lash. Eyelash extensions are very costly and take 2 hours to apply.

Cyanoacrylate was invented years ago and has many uses in the medical field. Recently, certain types of pharmaceutical grade cyanoacrylate, typically mixed with black pigments, have been used to bond individual eyelash extensions to the eyelashes. However, a pigmented cyanoacrylate has not been used as a stand alone, combination mascara and lash-lengthening coating application.

It is an object of this invention to provide a cosmetic composition that darkens, thickens, and lengthens eyelashes to which it is applied, and that remains in place for two weeks or more. It is a further object that the composition be comprised of a cyanoacrylate coating that bonds to the eyelash and cures to a waterproof state. It is another object of the invention to provide a method of applying, in less than 30 minutes, a cosmetic composition that darkens, thickens, and lengthens eyelashes. It is another object of the invention to provide a kit including the inventive composition and the requisite tools for performing the inventive method. Other objects and advantages of the present invention will become apparent from the following descriptions, taken in connection with the accompanying drawings, wherein, by way of illustration and example, an embodiment of the present invention is disclosed.

### SUMMARY OF THE INVENTION

A semi-permanent, lash-lengthening mascara comprises a cyanoacrylate coating mixed in a particular ratio with a discrete amount of micro- or nano-sized polytetrafluoroethylene (“PTFE”) fibers. The cyanoacrylate coating may comprise from about 70% to 100% pharmaceutical-grade cyanoacrylate, most preferably 75% to 85% cyanoacrylate, which has been distilled at least two times to remove impurities. The cyanoacrylate coating may further comprise one or more additives for rubber-toughening, thickening, coloring, and preserving the coating. Preferably, the additives are combined with the cyanoacrylate immediately upon distillation, while the cyanoacrylate is near its vaporization temperature. The PTFE fibers are preferably about one micron in width and irregularly shaped so that the fibers do not align neatly within the mascara. The PTFE fibers are mixed at a ratio of between about 1:1 to about 1:5 with the coating, most preferably between 1:3.33 and 1:4, thickening the coating and providing surface area that is bonded to the eyelash by the cyanoacrylate. The coating and PTFE fibers may be provided in separate containers and mixed immediately before application to the eyelashes, or the PTFE fibers may be cold-pressed into the coating and sealed in a waterproof container as an off-the-shelf pre-mixed product.

Methods of applying the inventive mascara include applying the mascara to the eyelashes within a few minutes of exposing the mixed mascara to air. In the preferred method, the lashes are first cleansed to remove debris, and one or more foam pads are adhered to the skin under the lash area to protect the skin during application. Preferably, a mascara wand is used to apply a polyvinyl primer to the lashes. The lashes may be curled if desired. A stationary or hand-held mister is held so that air and water mist is blown onto the eye area. The mixed, liquid mascara is loaded onto a mascara wand and applied to the lashes from base to tip, extending beyond the tip if additional length is desired. The application is repeated until the desired length and volume is achieved. The mister blows away fumes and cools the area to maintain comfort of the wearer, while the water mist provides a catalyst for the polymerization of the coating, speeding the curing process. The polyvinyl primer, preferably comprising over 50% water, may be applied immediately over the curing mascara to make the mascara cure even faster. Under proper application conditions, the inventive mascara cures in 5 minutes or less and lasts 2 to 3 weeks. The application process may further comprise removing the cured mascara, which requires a glue solvent, preferably having a base of gamma-Butyrolactone (“GBL”) or alternatively another solvent that is non-toxic in the amounts required to remove the mascara.

A kit is described, the kit containing the necessary compositions and tools for performing the inventive methods of applying the inventive mascara in the most efficient and effective manner. The kit includes known or specially designed tools for separating lashes, curling lashes, and applying compositions to lashes. The kit further includes the inventive mascara in separated or mixed form, primer, and remover. The kit further includes a mister designed for use in the inventive methods. The kit may further include adhesive foam pads for protecting the skin of the eye area, and one or more cleansing pads for cleaning the eye area and the tools.

### BRIEF DESCRIPTION OF DRAWINGS

FIG. 1 is a top view of tools and process to mix the adhesive to the dry powder microfibers.

FIG. 2 is a top view of mixing the adhesive with the dry powder microfibers.

FIG. 3 is a front view of the eye area with bottom eyelashes covered with gel pad.

FIG. 4 is a front view of the eye area with bottom eyelashes not covered by gel pad.

FIG. 5 is a front view of the process to apply the product to top eyelashes.

FIG. 6 is a front view of the process to apply the product to the bottom eyelashes.

FIG. 7 is a front view of the process of precision application of the product to the top eyelashes.

FIG. 8 is a front view of the process and tools used to remove the product from the eyelashes.

FIGS. 9 and 10 are top views of the applicator tools used in the invention.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention includes a semi-permanent, lash-lengthening mascara, methods of applying the mascara, and a kit containing the compositions and tools needed to perform the inventive methods of applying the inventive mascara. The inventive mascara is referred to herein as “the mascara,” although the mascara exhibits several properties not present in known mascaras. The most advantageous of these properties are that the mascara lengthens the lashes in addition to adding volume and color, and that the mascara cures in situ and lasts for two or more weeks.

Mascara Components

The mascara comprises a coating and an amount of micro- or nano-sized PTFE fibers, mixed in a particular ratio to achieve a desired viscosity, color, and curing time. The coating comprises primarily pharmaceutical-grade cyanoacrylate at between about 75% and 100% of the coating. The cyanoacrylate undergoes at least two distillation procedures, and further may receive precipitation agents for removing impurities, as is known in the art. The purity of the resulting cyanoacrylate allows it to cure at a reliable rate and exhibit less undesirable off-gassing than conventional cyanoacrylate products, such as industrial superglues. Preferably, the cyanoacrylate is non-blooming and has no odor. The most preferred cyanoacrylate is 2-methoxyethyl 2-cyanoacrylate, which has been shown to create a strong and flexible bond to keratin fibers. The cyanoacrylate may further comprise a stabilizer such as polymethylmethacrylate at 1% to 15% weight.

The coating may further comprise one or more additives for achieving certain desired characteristics. The additives are preferably added to the cyanoacrylate while the cyanoacrylate is still heated from its distillation process, most preferably within minutes after the cyanoacrylate transitions into liquid form, to ensure that the additives dissolve or achieve a stable suspension within the cyanoacrylate. Additives may affect characteristics such as color, strength, viscosity, and shelf life, among others. The additives suggested below are favored but not limiting.

A pigment may be added to darken or color the coating. Preferably, carbon black is added in an amount of about 0.05% to 0.1%, most preferably 0.09%, of the coating. Brown pigment may be used instead or in addition to maintain a natural look, or other colors may be added to achieve a striking colored effect.

An elastomeric compound may be added to serve as a substrate to the cyanoacrylate, strengthening the coating. The elastomeric compound may be a synthetic rubber, such as synthetic acrylic rubber. Naturally-occurring or organic rubbers cannot be used due to its lack of heat resistance—it cannot be added at high temperature—and further due to its typical impurity. Synthetic rubber may be added at up to about 10% of the coating, preferably about 6% to achieve the desired additional tensile strength without over-diluting the adhesive properties.

A thickening agent may be added to achieve a desired viscosity. The desired viscosity is commensurate with a thin, spreadable paste exhibiting a higher degree of controllability than a non-thickened cyanoacrylate typically exhibits. The desired viscosity is between about 500 and 1200 cP, most preferably around 750 cP to approximate the consistency of traditional mascara. The thickening agent is preferably PTFE, added in either a micro- or nano-sized solid fiber state at between about 5% to 15%, most preferably about 10%, of the coating.

One or more preservatives may be added to the coating to improve its shelf life. In particular, resistance to oxidation is desirable to prevent drying or clouding of the coating while it is being stored. Bacteriostatic butyl esters and antioxidants such as butylated hydroxyanisole and butylated hydroxytoluene may be added in very small concentrations to preserve the coating without overexposing it to moisture. Preferably, both of these antioxidants are added at about 0.09% to 0.1% of the coating. When added immediately following the distillation process, these antioxidants also help eliminate free radicals and other impurities in the cyanoacrylate.

The PTFE fibers are mixed with the coating and serve two functions. First, the PTFE fibers thicken the coating to the final application consistency. Second, the PTFE fibers provide a solid bonding surface to the cyanoacrylate in the coating. As it cures, the cyanoacrylate creates crosslinked polymerization between the PTFE fibers and the keratinous fibers of the eyelash, bonding the mascara to the lash. Successive coatings of the mascara, applied as described below, then bond to the cured layer of mascara underneath as the cyanoacrylate polymerizes between the PTFE fibers and the cured layer. This polymerization allows “lengthening” of the lash by drawing out the mascara as described below.

To achieve their functions, the PTFE fibers may be any shape, such as spherical, cubic, or irregular, and may be any size from several nanometers up to about 200 micrometers in average particle size. Preferably, the PTFE fibers are irregularly shaped with an average size of 40 to 90 micrometers. The irregular shape prevents the PTFE fibers from stacking neatly next to each other, as fibers of spherical or other uniform shape inherently do. The different alignment of stacked fibers is evident in the fibers' dry form: a jar full of spherical or cubic fibers looks like salt, and the fibrous “powder” feels slick between the fingers; a jar of irregular fibers looks fluffy, like flour, and is significantly less lubricious between the fingers. When added to the coating, the irregular fibers resist sliding by each other, resulting in a thicker mascara and providing increased surface area for bonding when compared to the same amount of uniformly-shaped fibers. It has also be shown by experimentation that the irregular fibers are preferred for the pre-mixed mascara described below: while uniformly-shaped fibers settle out of the mixture over time, requiring the mascara container to be shaken vigorously before use, the irregular fibers do not settle out and the mascara is dispensed from the container with substantially uniform consistency without shaking. The preferred particle size allows the PTFE fibers to remain substantially uniformly, on average, dispersed within the coating. This uniformity gives the mascara a natural texture that does not feel heavy on the lashes, while also adding apparent volume to the lashes.

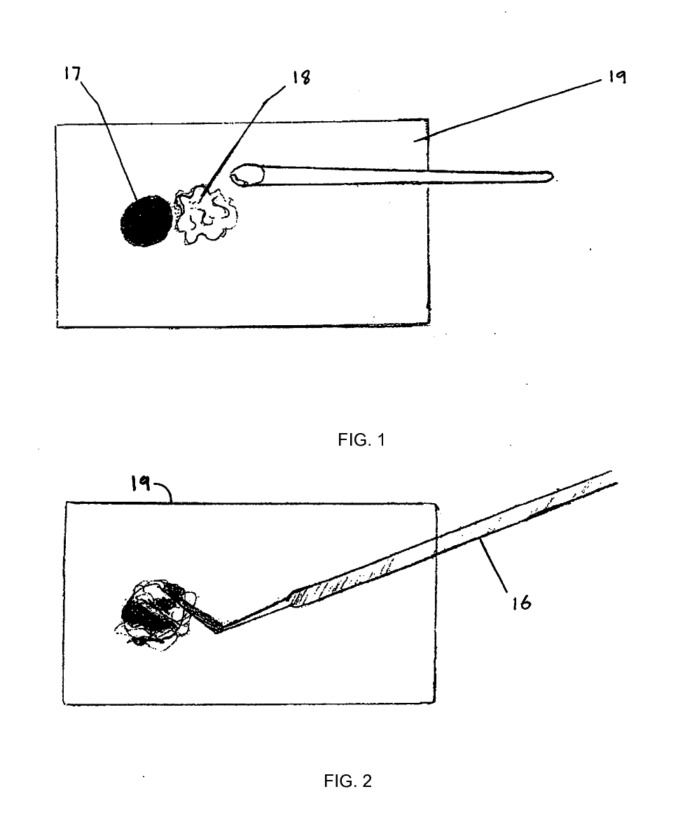

The coating and PTFE fibers are mixed together to achieve the desired consistency for application of the mascara. The ratio of PTFE fibers to coating may be between about 1:1 and about 1:5, with the resulting mascara being increasingly thick as the ratio approaches 1:1. Preferably, the ratio is between about 1:3.33 and about 1:4, giving a mascara with sufficient thickness and PTFE fiber bonding surface, and with a low enough viscosity to stick to and spread along individual lashes and look similar to traditional mascara. The mascara may be mixed onsite immediately prior to application, as illustrated in FIGS. 1 and 2 and described below, or the mascara may be premixed and stored in an airtight and watertight container. To premix the mascara, preferably the PTFE fibers are cold-pressed into the coating. To cold-press the PTFE fibers, the desired amount of PTFE fibers is added to the coating in a vacuum-sealed container at room temperature, and the container is vigorously shaken for several minutes to disperse the PTFE fibers within the coating.

Application Methods

The mascara may be applied by the wearer to her own lashes, but preferably it is applied by a salon professional who has been trained in the application methods. Training is preferred because the components may irritate the skin or eyes if improperly applied, and because the methods take about 30 minutes and it is therefore best to correctly apply the mascara without need for cleaning up mistakes.

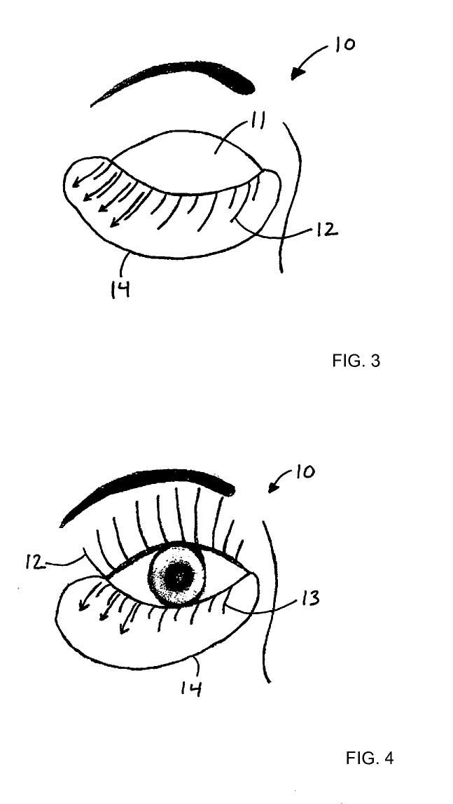

Referring to FIGS. 1-7, the professional first obtains the mixed mascara to be applied. In one embodiment, the mascara is premixed and the professional simply squeezes the mascara out of a tube onto an applicator, such as a mascara wand 15 or pick applicator 16. In another embodiment, the coating 17 is mixed with the PTFE fibers 18 to the correct consistency on a non-reactive slab 19, made of glass or stainless steel, immediately before application.

The professional cleans the eye area 10 and lashes 12, 13. The professional then places protective medical tape or gel eye pads 14 on the skin beneath the bottom lashes 13 to protect the skin. Preferably, the gel eye pads 14 have a mild adhesive on one side and are particularly shaped as illustrated to fit under the eye. Next, the lashes 12, 13 are curled with an eyelash curler 25, if desired. If necessary, the lashes 12, 13 may be separated from each other to prepare them for receiving the mascara. An improved separator 40 is provided, having a stainless steel construction for bacterial resistance and cleanability, and uniformly-sized and—spaced lash-separating large grooves 41 on one end for top lashes 12 and small grooves 42 on the opposing end for bottom lashes 13.

The professional may prime the lashes 12, 13 to receive the mascara. The preferred primer comprises a polyvinyl base, preferably polyvinylpyrrolidone (“PVP”) combined with (poly)acrylate in equal parts and suspended in a water and ethanol solution. The primer prepares the surface of the lash and adds structure and volume to the lash to improve adhesion of the coating. The primer may further comprise a pigment that colors the lashes 12, 13 when the primer is applied. The mascara, which may be made semi-translucent, allows some of the color in the primer to show through, creating a faintly colored or iridescent effect on the lashes 12, 13. The primer is allowed to dry for at least 20 seconds before continuing.

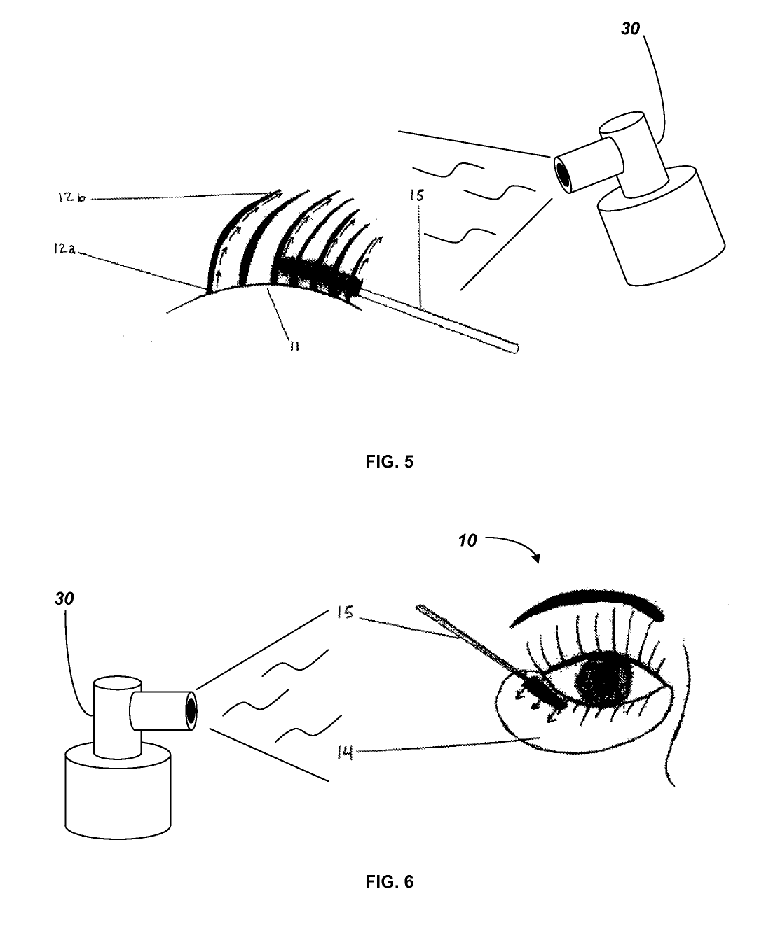

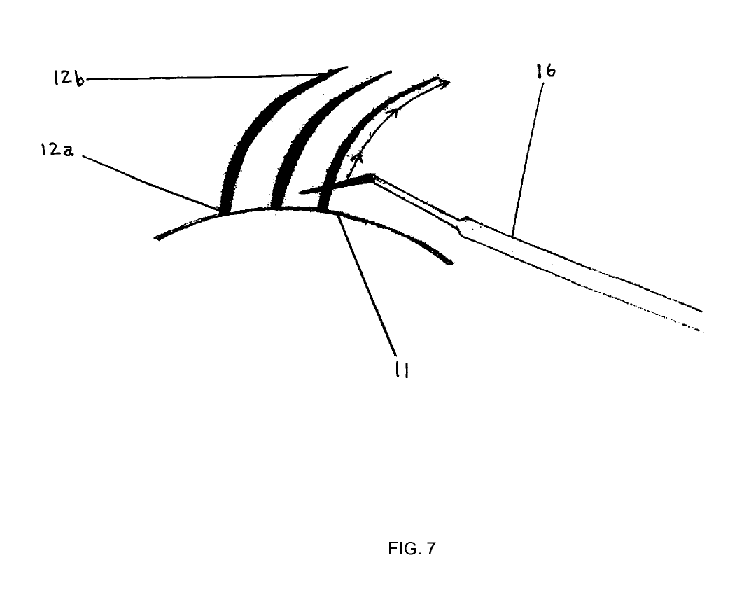

The professional positions the mister 30 so that it blows a light mist onto the eye area 10 Preferably, the mister 30 outputs between about 0.5 and about 1.5 milliliters of atomized water per minute, so that the mist provides hydration for the curing of the coating, but does not overly wet the eye area 10. The mister 30 may be handheld by the wearer or an assistant, or the mister 30 may be a stationary or table-top mister, such as a nebulizer filled with distilled water. The professional loads a stainless-steel pick applicator 16 or mascara wand 15 with mascara and applies the mascara to each individual eyelash, starting with the top lashes 12. The mascara is applied by stroking lashes evenly from lash base 12a to lash tip 12b, and extending beyond the lash tip 12b as allowed by the viscosity of the mascara. At the preferred viscosity, the mascara may be rolled one to two millimeters past the lash tip 12b on each stroke. The mascara wand 15 allows application to multiple lashes at once, see FIGS. 5-6, while the pick applicator 16 allows precision application to single lashes, see FIG. 7. The application is repeated until all eyelashes 12 on the top lid 11 are coated and lengthened as desired. Typically, no more than two coats are needed. Fumes from the curing cyanoacrylate are blown out of the wearer's face by the mister 30. The cyanoacrylate draws moisture out of the light mist, hastening polymerization so that the mascara cures quickly.

While the mascara is curing, the separator 40 may be used again to remove excess mascara and even out the application. Another layer of primer may be applied on top of the mascara. The water in the primer causes the mascara to cure almost instantly, while the polymeric base bonds to the coating and serves as a sealant to extend the life of the mascara. The mascara, thus sealed, also retains the curl of the lashes 12, if any. Further, the primer may be colored as described above, lending a significant colorized effect to the lashes 12 according to the concentration of the pigments in the primer.

After about five minutes, the professional applies the mascara to the bottom lashes 13 in the presence of the light mist, and then smoothes the lashes 13 and removes excess mascara as described above. Again, the mascara on the bottom lashes 13 may be instantly cured by applying a layer of primer, which may be colored. The skin-protecting pads 14 are removed after the bottom lashes 13 have dried for about five minutes.

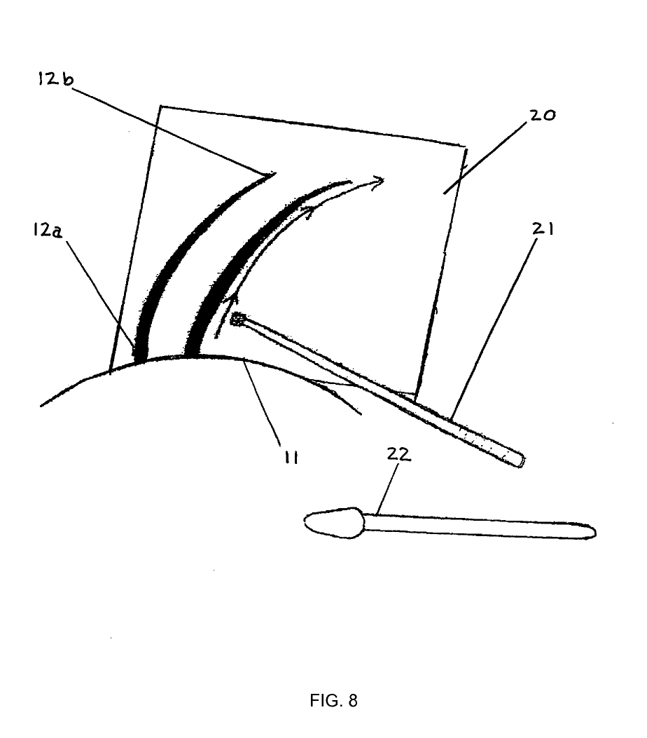

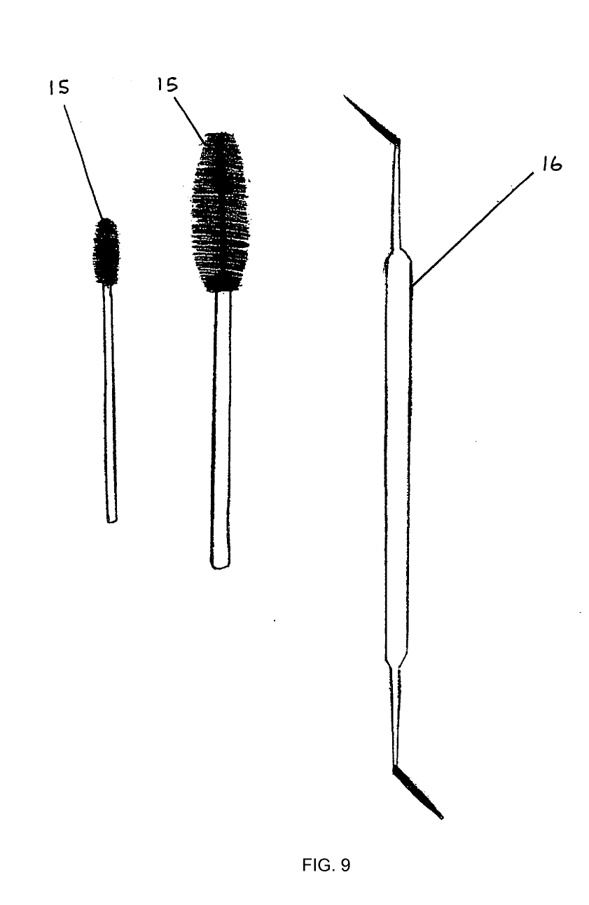

The resulting mascara has a similar texture to traditional mascara but is waterproof and smear-proof, and will never run. An application may last 2-3 weeks or more. To enhance the life of the application even further, a layer of primer may be reapplied as desired to protect the application. The preferred primer is water-soluble, and so will wash off in the shower. A colored primer may therefore be applied over the mascara for a single-day wear as desired. The mascara may be professionally removed, or it will simply wear off in 2-3 weeks on its own. A removing solvent must be able to dissolve cured cyanoacrylate. The preferred remover comprises about 95% gamma-Butyrolactone with about 3% dimethylketone and about 2% ethyl alcohol. Referring to FIG. 8, to remove the coating, the professional holds a non-woven 2×2 pad 20 under or behind the lashes 12, 13. Using a micro brush 21 or foam shadow applicator 22, the professional applies the liquid remover by pressing lashes 12, 13 brushing on the remover with a non-reactive, preferably silicon, cleansing brush 51 to release the coating from the lashes 12, 13 until all coating is removed. The lashes 12, 13 are then rinsed and cleansed with warm water and soap.

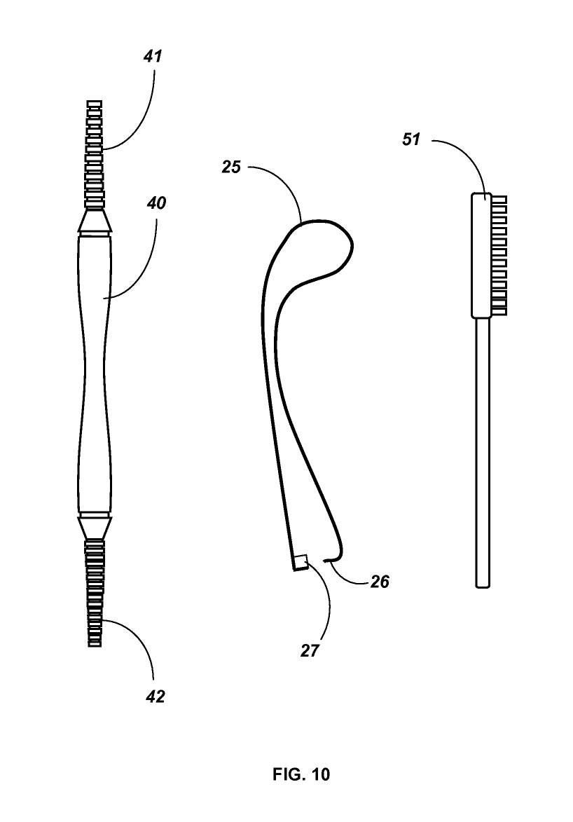

FIGS. 9 and 10 illustrate tools for performing the inventive methods of applying the inventive mascara. Some of the tools, such as mascara wands 15 of varying sizes, pick applicator 16, micro brush 21, shadow applicator 22, silicon cleansing brush 51, are commonly used in eye makeup applications. The illustrated eyelash curler 25 is an improvement over existing curlers in that the metal tooth 26 presses the lashes 12, 13 into a resilient polymer block 27, which is preferably made of silicon so that the tooth 26 presses into and deforms the block 27, thus curling the lashes 12, 13 around the tooth 26 within the block 27. The illustrated separator 40 is an improvement over existing separating tools in that it provides grooves for different-sized lashes on a single pen-like tool, which is stainless steel for ease of cleaning. The illustrated and described tools may be included with the mister 30, the components of the mascara, the primer, and the remover, in a professional kit provided to professionals after they are trained in the application methods. The kit may further comprise eye pads 14 and non-woven pads 20 to provide everything needed to perform the application.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A semi-permanent mascara composition comprising:
a) a coating comprising a cyanoacrylate that has been distilled two or more times; and
b) polytetrafluoroethylene (“PTFE”) fibers each having a size of 200 micrometers or less.

2. The composition of claim 1 wherein the coating further comprises a pigment.

3. The composition of claim 1 wherein the PTFE fibers have irregular shape.

4. The composition of claim 3 wherein the PTFE fibers have a size of between 40 and 90 micrometers.

5. The composition of claim 4 wherein the composition remains in place for about 2-3 weeks after application to eyelashes.

6. The composition of claim 1 wherein the coating further comprises an amount of PTFE up to 15% of the coating, the PTFE being added to the coating when the cyanoacrylate is near its vaporization temperature.

7. The composition of claim 6 wherein the coating further comprises an amount of synthetic rubber up to about 7% of the coating, the synthetic rubber being added to the coating when the cyanoacrylate is near its vaporization temperature.

8. A method of applying a semi-permanent mascara, the method comprising:
a) applying a composition to one or more top eyelashes, the composition comprising:
i. a coating comprising a cyanoacrylate that has been distilled two or more times; and
ii. PTFE fibers each having a size of 200 micrometers or less;
b) applying the composition to one or more bottom eyelashes; and
c) while applying the composition to the bottom eyelashes, using a mister to blow air and a list mist of water onto an eye area containing the top and bottom eyelashes.

9. The method of claim 8 further comprising cleansing the one or more eyelashes before applying the composition.

10. The method of claim 8 further comprising applying a primer to the one or more eyelashes after cleaning the eyelashes and before applying the composition.

11. The method of claim 10 wherein the primer is a polyvinyl primer.

12. The method of claim 8 further comprising applying a primer over the composition to hasten the curing process of the composition, the primer comprising water and a polyvinyl base.

13. The method of claim 8 further comprising curing the composition for about 2-5 minutes after application of the composition to the one or more eyelashes.

14. The method of claim 8 further comprising allowing the composition to remain in place one the one or more eyelashes for about 2-3 weeks after application.

15. A method of applying a semi-permanent mascara, the method comprising:
a) mixing a coating comprising cyanoacrylate and polytetrafluoroethylene fibers to form a composition;
b) within 2-5 minutes of mixing, applying the composition to one or more eyelashes;
c) using a mister, blowing air and a light mist of water onto an eye area containing the one or more eyelashes; and
d) curing the composition for about 2-5 minutes after application of the composition to the one or more eyelashes.

16. The method of claim 15 further comprising cleaning the one or more eyelashes before applying the composition.

17. The method of claim 16 further comprising applying a primer to the one or more eyelashes after cleaning the eyelashes and before applying the composition.

18. The method of claim 17 further comprising applying the primer over the composition in order to cure the composition substantially instantly.

19. The method of claim 18 wherein the primer comprises water and a polyvinyl base.

20. The method of claim 19 wherein:
a) the cyanoacrylate comprises 2-methoxyethyl 2-cyanoacrylate and comprises 75%-84% of the coating;
b) the coating further comprises PTFE at about 10% of the coating and synthetic rubber at about 6% of the coating; and
c) the PTFE fibers are irregularly shaped with an average size of 40-90 micrometers.
